(19) United States
(12) Patent Application Publication
(10) Pub. No.: US 2026/0287569 A1
(43) Pub. Date: Sep. 24, 2026

(54) SYSTEM AND METHOD FOR DETERMINING ONE OR MORE DEFECTS IN ONE OR MORE COMPONENTS

(71) Applicant: Panasonic Intellectual Property Management Co., Ltd., Osaka (JP)

(72) Inventors: ZONG SHENG TANG, Singapore (SG); PHILIP HANN YUNG LEE, Singapore (SG); ARIEL BECK, Singapore (SG); SAHIM YAMAURA, Singapore (SG); TOSHIHIRO SAKAMOTO, Osaka (JP); HIROMU KITAJIMA, Hyogo (JP)

(21) Appl. No.: 19/083,487

(22) Filed: Mar. 19, 2025

(51) Int. Cl. G01N 33/00; G06F 3/04847; G06T 11/20

(52) U.S. Cl. CPC G01N 33/007; G06F 3/04847; G06T 11/26; G06T 2200/24

(57) ABSTRACT

A method for determining one or more defects in one or more components is disclosed. The method includes receiving a first user input indicating a selection of a pre-stored application domain, receiving a second user input via a graphical user interface (GUI), and receiving a third user input via the GUI. Further, determining a machine learning (ML) model based on the first user input, the second user input, and the third user input. Further, generating component characteristics, using the determined ML model. Further, determining the one or more defects in the one or more components based on correlating the component characteristics with the threshold value.

### TECHNICAL FIELD

The present invention generally relates to a component inspection, and more particularly relates to a method and a system for determining one or more defects in one or more components.

### BACKGROUND

The information in this section merely provides background information related to the present disclosure and may not constitute prior art(s) for the present disclosure.

Component detection systems, especially, odorant detection, are widely used in various industries, for instance, food processing industry, textile manufacturing industry, drug industry, etc., for environmental monitoring, food safety, industrial quality control, and hazardous gas detection. The component detection systems typically rely on sensors that capture signals, e.g., chemical or physical signals, from the surrounding environment and analyse the captured signals to determine the presence and concentration of specific compounds in the environment at an industrial site.

Traditional component detection systems utilize gas chromatography, mass spectrometry, or electrochemical sensors, which provide quantitative outputs indicative of detected components. The quantitative outputs determine defect in components. Further, to improve efficiency of the component detection, machine learning (ML) and artificial intelligence (AI) models have been integrated into the traditional component detection systems, allowing for automated classification and judgement of the defect based on predefined thresholds of the quantitative outputs. Thus, the introduction of AI-driven detection has enabled real-time analysis and decision-making, reduced human intervention and improved accuracy.

Moreover, the traditional component detection systems commonly employ rule-based logic or pretrained AI models for processing sensor data and generating defect judgments. Some solutions allow users to define the predefined thresholds manually, while others rely on predefined standards for determining whether a detected component is within acceptable limits. In industrial applications, sensor health monitoring is often included to ensure reliability, where periodic calibration and performance validation are required. Existing graphical user interfaces (GUIs) provide visualization of detection results, enabling users to interpret quantitative outputs through charts, graphs, and defect classifications. Despite advancements in detection technologies, existing solutions related to the GUIs still face limitations in adaptability, automation, and user accessibility.

One of the primary challenges with current detection systems is the complexity of configuring the AI models and detection parameters for specific applications. Many solutions require extensive technical expertise to select an appropriate AI model, set detection threshold values, and calibrate sensors effectively. Additionally, sensor degradation over time leads to inaccurate defect judgments, yet most existing systems lack an integrated mechanism for dynamically assessing sensor health and adjusting detection models accordingly. Another challenge is the rigidity of predefined AI models, which may not be optimized for industry-specific requirements or evolving environmental conditions.

Therefore, there is a need for an alternative solution that may overcome above discussed limitations and provide an improved method and a system for determining one or more defects in one or more components.

The drawbacks, difficulties, disadvantages, or limitations of the conventional techniques explained in the background section are just for exemplary purposes and the disclosure would never limit its scope only such limitations. A person skilled in the art would understand that this disclosure and below mentioned description may also solve other problems or overcome the other drawbacks/disadvantages.

### SUMMARY

This summary is provided to introduce a selection of concepts, in a simplified format, that are further described in the detailed description of the invention. This summary is neither intended to identify essential inventive concepts of the invention nor is it intended for determining the scope of the invention.

According to an aspect of the present disclosure, a method for determining one or more defects in one or more components is disclosed. The method includes receiving, a first user input indicating a selection of a pre-stored application domain. The first user input is received via a graphical-user-interface (GUI). Further, the method includes receiving a second user input via the GUI. The second user input indicates a threshold value associated with an attribute for each of the one or more components. The method further includes receiving a third user input via the GUI. The third user indicates one or more external variables associated with an environment at an application site. The method further includes determining a machine learning (ML) model among a plurality of pre-stored ML models based on the first user input, the second user input, and the third user input. Further, the method includes generating component characteristics, using the determined ML model. The component characteristics indicate physical and chemical features of the one or more components present in the application site. The method further includes determining the one or more defects in the one or more components based on correlating the component characteristics with the threshold value. The one or more defects indicate deviation or anomaly in the attribute of each of the one or more components.

According to another aspect of the present disclosure, a system for determining one or more defects in one or more components is disclosed. The system includes a memory communicatively coupled with a processor. The processor is configured to receive, a first user input indicating a selection of a pre-stored application domain. The first user input is received via a graphical-user-interface (GUI). Further, the processor is configured to receive a second user input via the GUI. The second user input indicates a threshold value associated with an attribute for each of the one or more components. The processor is configured to receive a third user input via the GUI. The third user indicates one or more external variables associated with an environment at an application site. The processor is configured to determine a machine learning (ML) model among a plurality of pre-stored ML models based on the first user input, the second user input, and the third user input. Further, the processor is configured to generate component characteristics, using the determined ML model. The component characteristics indicate physical and chemical features of the one or more components present in the application site. The processor is configured to determine the one or more defects in the one or more components based on correlating the component characteristics with the threshold value. The one or more defects indicate deviation or anomaly in the attribute of each of the one or more components.

To further clarify the advantages and features of the present invention, a more particular description of the invention will be rendered by reference to specific embodiments thereof, which are illustrated in the appended drawings. It is appreciated that these drawings depict only typical embodiments of the invention and are therefore not to be considered limiting of its scope. The invention will be described and explained with additional specificity and detail in the accompanying drawings.

### BRIEF DESCRIPTION OF THE FIGURES

These and other features, aspects, and advantages of the present invention will become better understood when the following detailed description is read with reference to the accompanying drawings in which like characters represent like parts throughout the drawings, wherein:

FIG. 1A illustrates a schematic block diagram depicting an environment for the implementation of a system for determining one or more defects in one or more components, in accordance with an embodiment of the present disclosure;

FIG. 1B illustrates an exemplary scenario depicting a graphical representation of a graphical user interface (GUI) for odorant sensing detection operation, in accordance with an embodiment of the present disclosure;

FIG. 1C illustrates another exemplary graphical representation of the GUI for optimized component detection with auto-recommended industrial specific artificial intelligence (AI) model, in accordance with an embodiment of the present disclosure;

FIG. 2 illustrates a schematic block diagram of the system for determining the one or more defects in the one or more components, in accordance with an embodiment of the present disclosure;

FIG. 3 illustrates a process flow diagram depicting blocks for determining the one or more defects in the one or more components, in accordance with an embodiment of the present disclosure;

FIG. 4 illustrates a flowchart depicting an exemplary method for determining the one or more defects in the one or more components, in accordance with an embodiment of the present disclosure;

FIG. 5 illustrates a flowchart depicting sub-steps for generating component characteristics using a machine learning (ML) model, in accordance with an embodiment of the present disclosure;

FIG. 6 illustrates a flowchart depicting sub-steps for generating an alert in response to an estimated accuracy of the ML model, in accordance with an embodiment of the present disclosure; and

FIG. 7 illustrates a flowchart depicting sub-steps for determining defect in a generated output of the ML model, in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having the benefit of the description herein.

### DETAILED DESCRIPTION OF THE FIGURES

For the purpose of promoting an understanding of the principles of the invention, reference will now be made to the various embodiments, and specific language will be used to describe the same. It should be understood at the outset that although illustrative implementations of the embodiments of the present disclosure are illustrated below, the present invention may be implemented using any number of techniques, whether currently known or in existence. The present disclosure is not necessarily limited to the illustrative implementations, drawings, and techniques illustrated below, including the exemplary design and implementation illustrated and described herein, but may be modified within the scope of the present disclosure.

It will be understood by those skilled in the art that the foregoing general description and the following detailed description are explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

It is to be understood that as used herein, terms such as, “includes,” “comprises,” “has,” etc. are intended to mean that the one or more features or elements listed are within the element being defined, but the element is not necessarily limited to the listed features and elements, and that additional features and elements may be within the meaning of the element being defined. In contrast, terms such as, “consisting of” are intended to exclude features and elements that have not been listed.

The embodiments herein and the various features and advantageous details thereof are explained more fully with reference to the non-limiting embodiments that are illustrated in the accompanying drawings and detailed in the following description. Descriptions of well-known components and processing techniques are omitted to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

As is traditional in the field, embodiments may be described and illustrated in terms of blocks that carry out a described function or functions. These blocks, which may be referred to herein as units or modules or the like, are physically implemented by analog or digital circuits such as logic gates, integrated circuits, microprocessors, microcontrollers, memory circuits, passive electronic components, active electronic components, optical components, hardwired circuits, or the like, and may optionally be driven by firmware and software. The circuits may, for example, be embodied in one or more semiconductor chips, or on substrate supports such as printed circuit boards and the like. The circuits constituting a block may be implemented by dedicated hardware, by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the invention. Likewise, the blocks of the embodiments may be physically combined into more complex blocks without departing from the scope of the invention.

The accompanying drawings are used to help easily understand various technical features and it should be understood that the embodiments presented herein are not limited by the accompanying drawings. As such, the present disclosure should be construed to extend to any alterations, equivalents, and substitutes in addition to those which are particularly set out in the accompanying drawings. Although the terms first, second, etc. may be used herein to describe various elements, these elements should not be limited by these terms. These terms are generally only used to distinguish one element from another.

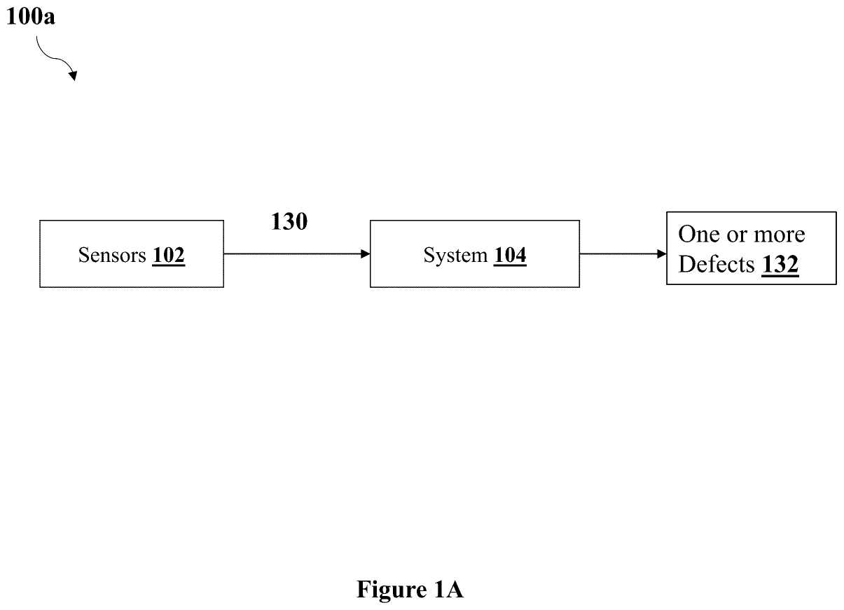

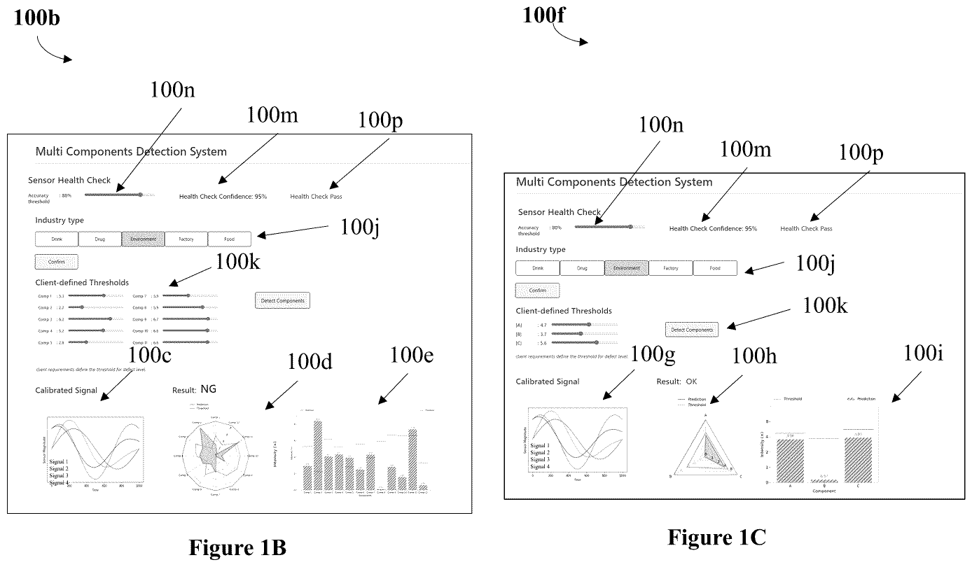

FIG. 1A illustrates a schematic block diagram depicting an environment 100a for the implementation of a system 104 for determining one or more defects 132 in one or more components, in accordance with an embodiment of the present disclosure.

In an embodiment, “one or more components” refers to physical and chemical elements of different tangible substances present at a physical location of an industry. For instance, in a petroleum industry, a “component” may be a gas, a chemical element. The component may have attributes that may determine the quality of the component. For instance, gas (component) has an odor intensity, therefore a threshold value of the intensity of the odor may determine a defect in the gas.

In an embodiment, “application site” refers to a physical or actual location or site of the industry (e.g., the industrial site). For example, food industry, beverage industry, drug industry, alcohol industry, environment industry, etc. The application site may be interchangeably used with “industry”, industrial site, industrial application site, physical location, etc. In an embodiment, “pre-stored application domain” or “application domain” may refer to a virtual location or a stored location over a graphical user interface (GUI). In an embodiment, the virtual location may correspond to a simulated location of a physical location. The pre-stored application domain may be selected by a user/an operator over the GUI. The pre-stored application domain or the application domain or a domain may be used interchangeably throughout the disclosure.

In an embodiment, the environment 100a may include a plurality of sensors 102 adapted to detect stimulant data 130 associated with the one or more components present at the application site. The plurality of sensors 102 may be installed at multiple locations at the application site. In an embodiment, the plurality of sensors 102 may alternatively be referred to a sensor 102 within the scope of the present disclosure.

In an embodiment, the plurality of sensors 102 refers to physical devices that are installed at the application site. The plurality of sensors 102 are adapted to detect data related to the one or more components present within the application site. The data detected by the plurality of sensors 102 may be referred to as the stimulant data 130. Further, the stimulant data 130 related to the one or more components may be analysed and processed with respect to threshold values defined by a user, for example, an operator. In an embodiment, when the stimulant data 130 is above or below the threshold values, the component may be categorized to be defective or have a defect.

The environment 100a may further include the system 104 configured to determine the one or more defects 132 in the one or more components. The system 104 may be communicatively coupled to the sensor 102. The system 104 may be configured to estimate the accuracy of a machine learning (ML) model based on the stimulant data 130. The ML model may be configured to predict characteristics of the one or more components. Further, the system 104 may be configured to generate an alert in response to the estimated accuracy of the ML model. The system 104 may be implemented in an exemplary scenario as illustrated in FIG. 1B.

FIG. 1B illustrates the exemplary scenario depicting a graphical representation 100b of the GUI for odorant sensing detection operation, in accordance with an embodiment of the present disclosure. In an embodiment, the GUI may be referred to as the graphical representation 100b and may be used interchangeably hereafter.

As illustrated, the GUI 100b displays an accuracy threshold indicated by 100n as 80%. The accuracy threshold 100n is referred to as a predefined accuracy threshold defined by user. Further, the GUI 100b displays accuracy estimation of the ML model by a sensor health check model, for example, the sensor health check model estimates that the accuracy of the ML model is 95%, as indicated by 100m. Further, the GUI 100b displays a health check status indicated by 100p, for example, pass or fail. The health check status is determined by comparing estimated accuracy of the ML model by the sensor health check model with the accuracy threshold 100n. for example, the estimated accuracy of the ML model is 95% which is more than the accuracy threshold of 80%, therefore, the health check status displayed by the GUI is Pass.

In the illustrated embodiment, the GUI may be configured to display the graphical representation 100b including pre-stored application domains. Further, the user may interact with the graphical representation 100b for selecting at least one application domain. For example, the GUI 100b displays “industry type” indicated by 100j and prompts the user to select at least one industry type 100j from “alcohol”, “drink”, “drug”, environment”, “food”. The user selects “environment”. Further, the GUI prompts the user to confirm the selection of the industry type 100j, i.e., “environment”.

Further, the GUI 100b displays client-defined thresholds 100k, i.e., threshold values selected by the user, to detect an attribute in the selected industry type, i.e., environment. For example, the attribute refers to properties of the one or more components, such as, gas is a component, and the attribute of gas includes odor. Further, the one or more components includes e.g., helium gas, methane gas, carbon dioxide gas, etc., in the environment. For the sake of brevity, 11 components are considered, as illustrated in the FIG. 1B. The user selects the threshold values for each of the 11 components as comp 1: 5.3, comp 2: 2.2, comp 3: 6.2, comp 4: 5.2, comp 5: 2.8, comp 7: 3.9, comp 8: 5.9, comp 9: 6.7: comp 10: 6.6, comp 11: 6.6. The threshold values are selected by the user and may be referred to as the client-defined thresholds 100k, which indicate that the odor of the comp 1 is acceptable when the odor is below 5.3. Further, the GUI 100b may display external variables related to the application site and may prompt the user to select one or more external variables, such as temperature, pressure, etc. In an embodiment, any variation in the external variables may affect the stimulant data of the plurality of sensors 102. Therefore, the external variables may be selected to compensate for the changes in the stimulant data of the plurality of sensors 102.

The system 104 may determine the ML model based on the selected industry type, the client-defined thresholds 100k and the one or more external variables. The ML model generates or predicts characteristics of the component. The characteristics predicted by the ML model are correlated with the client-defined thresholds 100k and a result related to acceptance or rejection of a component is displayed over the GUI 100b.

Further, GUI 100b displays graphic visualizations of intensities in visual formats. For example, GUI 100b displays a chart 100c depicting sensor magnitude versus time(s) for a plurality of channels, indicated as signal1 to signal4. The chart indicates that the variation in sensor magnitude for the plurality of channels is randomly distributed. Further, the GUI 100b displays a graph 100d of the components 1-12, between predicted values or referred to as augmented data and the client-defined thresholds 100k. Further, the GUI 100b displays a bar chart 100e depicting the components 1-12, with respect to intensity (k) of the attribute of the component. The bar chart indicates the client-defined thresholds as a datum line, input value, predicted value for real data and augmented data. Therefore, the acceptance and rejection of the component is based on the result displayed over the GUI 100b. For instance, for a component 2, threshold value for odor intensity is 2.2 and the predicted odor intensity is 8.36. Therefore, the comp 1 may be flagged as rejected or not acceptable or not good (NG) as displayed over the GUI 100b.

FIG. 1C illustrates another exemplary graphical representation 100f of the GUI for optimized component detection with auto-recommended industrial specific artificial intelligence (AI) model, in accordance with an embodiment of the present disclosure. The graphical representation 100f may be referred to as GUI 100f and may be interchangeably used in this exemplary embodiment. The GUI 100f may be configured for optimized component detection with an auto-recommended industrial specific AI model.

In another exemplary embodiment, multi component detection is performed, over the GUI 100f. In the multi component detection, a selection of pre-defined application domains appears. For example, industry types 100j “drink”, “drug”, “environment”, “factory”, and “food”. Further, the user selects one or more industry types 100j and the GUI 100f prompts a confirmation regarding the selection.

Further, the GUI 100f prompts to input the client-defined thresholds 100k for the one or more components. For example, the user sets a threshold for each detected gas. The user selects threshold values for the one or more components as A: 4.7, B: 3.7, and C: 5.6. The client-defined thresholds 100k indicate that the attributes, for example, intensity, force, sound, etc., of the one or more components are acceptable when the odor is below A: 4.7, B: 3.7, and C: 5.6. In an alternate embodiment, as described earlier, the GUI 100f may display external variables related to the application site and may prompt the user to select one or more external variables, such as temperature, pressure, etc.

Further, the ML model is automatically determined based on the provided client-defined thresholds 100k. Further, the component characteristics of the one or more components include predicted intensity. The predicted intensity is displayed in a radar plot and bar chart over the GUI 100f, as described earlier.

As illustrated, the GUI 100f displays that the sensor health check model has estimated the accuracy of the ML model as 80%, as indicated by 100m, based on the stimulant data 130, i.e., sensor data. In this case, the user defines the predefined accuracy threshold for each of the plurality of sensors 102 installed at the application site. In another case, the sensor health check model may estimate that the ML model is having an accuracy of less than 80%, then in this case, the sensor health check had failed.

The GUI 100f displays a graph 100g depicting sensor magnitude versus time in the plurality of channels, e.g., signal 1 to signal 4. The graph 100g shows variations in the sensor magnitude over time. The accuracy of the plurality of sensors 102 varies over time. Further, the GUI 100f displays a ternary graph 100h illustrating variation in predicted value and threshold value for each component, i.e., A, B, C. Further, the GUI 100f displays a bar chart 100i of the components with respect to intensity (x). The bar chart 100i indicates, for example, for component A, the threshold value for odor intensity is 4.56, however the predicted odor intensity is 3.56. therefore, the component A is flagged as acceptable or good.

The present disclosure may be explained in detail in the below paragraphs of the description.

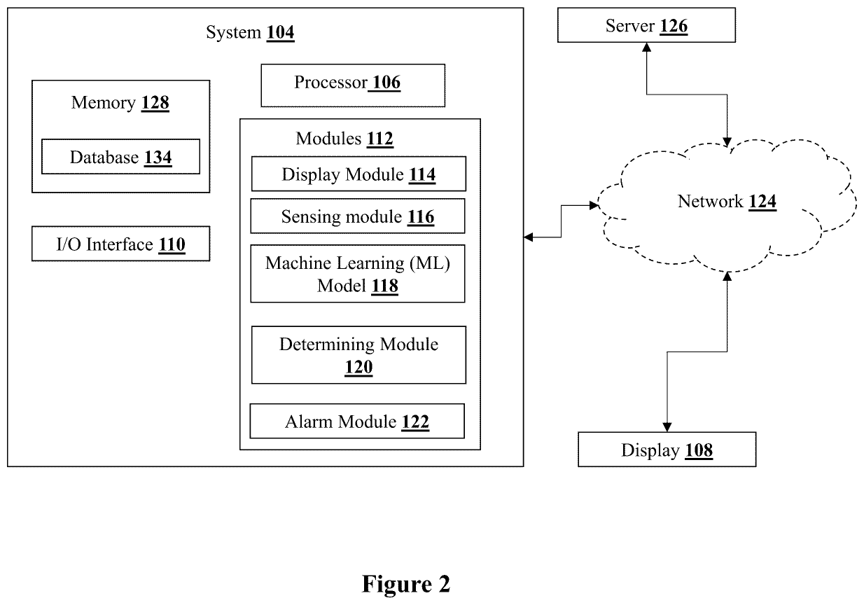

FIG. 2 illustrates a schematic block diagram of the system 104 for determining the one or more defects 132 in the one or more components, in accordance with an embodiment of the present disclosure.

In an embodiment, the system 104 may include a memory 128 including a database 134, a processor 106 communicatively coupled with the memory 128, a display 108, an Input/Output (I/O) interface 110, and a plurality of modules 112. In an embodiment, the system 104 may be implemented by a User Equipment (UE). In a non-limiting example, the UE may be a smartphone, a laptop computer, a desktop computer, a Personal Computer (PC), a notebook, a tablet, or a smartwatch.

In another embodiment, the system 104 may be a cloud-based system, that may include a server 126, specifically a cloud server. In yet another embodiment, the system 104 may be implemented by a combination of the UE and the server 126 via a network 124. In an embodiment, the system 104 may be implemented through the GUI of the UE. In an embodiment, the display 108 may also be referred to as the GUI 100b.

In an embodiment, the system 104 may be configured to determine defect in the component, estimate accuracy of the ML model, monitor sensor health and generate defect judgement of the component based on the accuracy of the ML model and sensor health check. In an implementation, the ML model may be determined based on various inputs received by the system 104 through the display 108. The various inputs may be pre-stored in the memory 128 and the user may be adapted to input as required.

In one embodiment, the memory 128 is configured to store instructions executable by the processor 106. In one embodiment, the memory 128 communicates via a bus within the system 104. The memory 128 includes but is not limited to, a non-transitory computer-readable storage media, such as various types of volatile and non-volatile storage media including, but not limited to, random access memory, read-only memory, programmable read-only memory, electrically programmable read-only memory, electrically erasable read-only memory, flash memory, magnetic tape or disk, optical media and the like. In one example, the memory includes a cache or random-access memory (RAM) for the processor 106. In alternative examples, the memory 128 is separate from the processor 106 such as a cache memory of a processor, the system memory, or other memory. The memory 128 is an external storage device or the memory 128 is for storing data. The memory 128 is operable to store instructions executable by the processor 106. The functions, acts, or tasks illustrated in the figures or described are performed by the programmed processor for executing the instructions stored in the memory 128. The functions, acts, or tasks are independent of the particular type of instruction set, storage media, processor, or processing strategy and may be performed by software, hardware, integrated circuits, firmware, micro-code, and the like, operating alone or in combination. Likewise, processing strategies include multiprocessing, multitasking, parallel processing, and the like.

As a non-limiting example, the processor 106 may be a single processing unit or a set of units each including multiple computing units. The processor 106 may be implemented as one or more microprocessors, microcomputers, microcontrollers, digital signal processors, central processing units, state machines, logic circuitries, and/or any devices that manipulate signals based on operational instructions (computer-readable instructions) stored in the memory 128. Among other capabilities, the processor 106 may be configured to fetch and execute computer-readable instructions and data stored in the memory 128. The processor 106 includes one or a plurality of processors. The plurality of processors is further implemented as a general-purpose processor, such as a central processing unit (CPU), an application processor (AP), or the like, a graphics-only processing unit, such as a graphics processing unit (GPU), a visual processing unit (VPU), and/or an AI-dedicated processor such as a neural processing unit (NPU). The plurality of processors controls the processing of the input data in accordance with a predefined operating rule or an artificial intelligence (AI) model stored in the memory 128. The predefined operating rule or the AI model is provided through training or learning.

Further, the processor 204 may be implemented as one or more microprocessors, microcomputers, microcontrollers, digital signal processors, central processing units, state machines, logic circuitries, and/or any devices that manipulate signals based on operational instructions. Among other capabilities, the processor 204 may be configured to fetch and execute computer-readable instructions and data stored in the memory 208 and/or the plurality of modules 114.

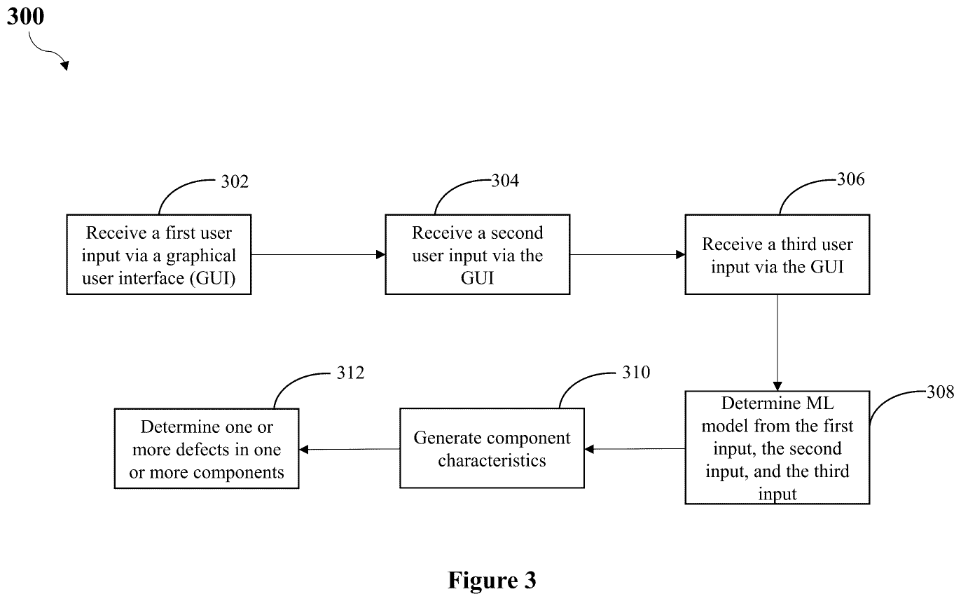

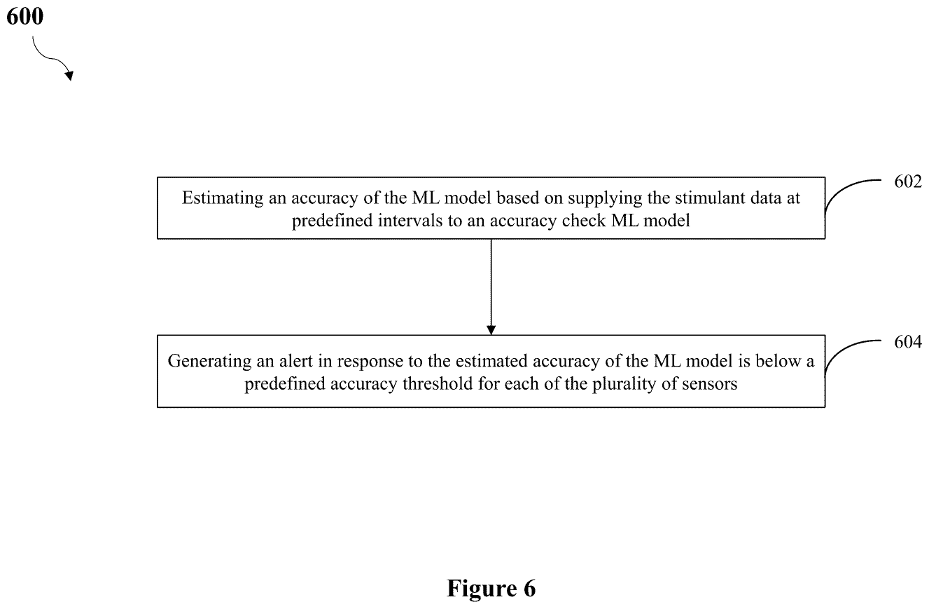

The plurality of modules 112 may include a display module 112, a sensing module 114, a ML module 116, a determining module 118, and an alarm module 120. In an embodiment, the display module 112, the sensing module 114, the ML module 116, the determining module 118, and the alarm module 120 may be in communication with each other. The plurality of modules 112 are described in greater detail in conjunction with FIGS. 3-5 along with exemplary use-cases in conjunction with FIGS. 6-8.

The processor 106 may be disposed in communication with one or more input/output (I/O) devices via the Input/Output (I/O) interface 110. The I/O interface 110 employs communication code-division multiple access (CDMA), high-speed packet access (HSPA+), global system for mobile communications (GSM), long-term evolution (LTE), WiMax, and the like, etc. In another embodiment of the present invention, the I/O interface 110 employs ethernet, industrial wireless Local Area Network (LAN), Process Field Bus (PROFIBUS), Actuator Sensor (AS) Interface, and the like.

The plurality of modules 112 may include the one or more instructions (stored in a memory 128) that may be executed to cause the system 104, in particular, the processor 106 of the system 104, to perform the one or more functions/methods, as discussed here in the present disclosure. In one embodiment, the plurality of modules 112 may be implemented at least in part as hardware, which may work in conjunction with the instructions to perform the functions/methods discussed herein.

As shown, the display 108 may include a Liquid Crystal Display (LCD), an Organic Light-Emitting Diode (OLED), a flat panel display, a solid-state display, a Cathode Ray Tube (CRT), a projector, a printer or other now known or later developed display device for outputting determined information. The display 108 may act as an interface for the user to see the functioning of the processor 106, or specifically as an interface with the software stored in the memory 128.

The present invention contemplates a computer-readable medium that includes memory 128 having executable instructions responsive to a propagated signal so that a device connected to the network can communicate voice, video, audio, images, or any other data over the network 124. Further, the instructions may be transmitted or received over the network 124 via the I/O interface 110 or referred to as a communication interface or port. The communication interface may be a part of the processor 106 or maybe a separate component. The communication interface may be created in software or maybe a physical connection in hardware. The communication interface may be configured to connect with the network 124, external media, the display 108, or any other components in UE, or combinations thereof. The connection with the network 124 may be a physical connection, such as a wired Ethernet connection, or may be established wirelessly as discussed later. Likewise, the additional connections with other components of the UE may be physical or may be established wirelessly.

The network 124 may include wired networks, wireless networks, Ethernet AVB networks, or combinations thereof. The wireless network may be a cellular telephone network, an 802.11, 802.16, 802.20, 802.1Q, or WiMax network. Further, the network 124 may be a public network, such as the Internet, a private network, such as an intranet, or combinations thereof, and may utilize a variety of networking protocols now available or later developed including, but not limited to, TCP/IP based networking protocols. The UE may not be limited to operation with any particular standards and protocols. For example, standards for Internet and other packet-switched network transmissions (e.g., TCP/IP, UDP/IP, HTML, and HTTP) may be used.

In an embodiment, the system 104 may be implemented through the plurality of modules 112 and the processor 106 by receiving the various inputs and determining the defect in the component. The various inputs may include a first input, a second input, and a third input received by the processor 106 via the GUI. The GUI may be configured to achieve effective attribute detection, e.g., odorant detection, of the component tailored to a specific industry and needs.

FIG. 3 illustrates a process flow diagram depicting blocks for determining the one or more defects 132 in the one or more components, in accordance with an embodiment of the present disclosure.

At block 302, the display module 114 may be configured to receive the first user input via the GUI 100b. The first user input may include the selection of the pre-stored application domain. As discussed earlier, the pre-stored application domain or an application domain may be referred to the type of industry. Stated differently, the pre-stored application domain may include various types of industries pre-stored within the database 134 and the user may be prompted over the GUI 100b to select at least one type of industry. The various types of industries may include drug industry, food industry, beverage industry, environmental industry, etc.

At block 304, the display module 114 may be configured to receive the second user input via the GUI 100b. In an embodiment, the second user input may include the threshold value associated with the attribute for each of the one or more components. The attribute for the component may be based on the physical or chemical variation in the properties of the component. For instance, the attributes of a gas in a petroleum or beverage industry, may be odor intensity, flow rate, etc. Further, the attribute associated with the component may have the threshold value beyond which the component may be flagged as defective or acceptable. For example, the odor intensity of the gas is detected on a scale as 7.5 and the threshold value is 7.5. Further, the component, i.e., the gas, is defective when the value of odor intensity is equal to or greater than 7.5. In an exemplary embodiment, the component may have different threshold values for different attributes. The threshold values may be selected by the user based on industry type and standards.

At block 306, the display module 114 may be configured to receive the third user input via the GUI 100b. The third user input may include one or more external variables associated with the environment at the application site. In an embodiment, the application site may refer to a factory having the physical location, for example, a biscuit factory, a gas-power plant, a petroleum refinery, etc. The one or more external variables may include temperature, air pressure, etc., of the environment within the application site. The user may be adapted to input the one or more external variables based on the type of the industry selected and the threshold value provided. The one or more external variables may be selected to compensate for the changes in the stimulant data of the plurality of sensors 102.

At block 308, the determining module 120 may be configured to determine the ML model among a plurality of pre-stored ML models based on the first user input, the second user input, and the third user input. In an embodiment, the plurality of pre-stored ML models may be pre-trained artificial intelligence (AI) models that may be adapted to detect or determine industrial-specific attributes. The determined ML model may be configured to predict the one or more defects in the one or more components.

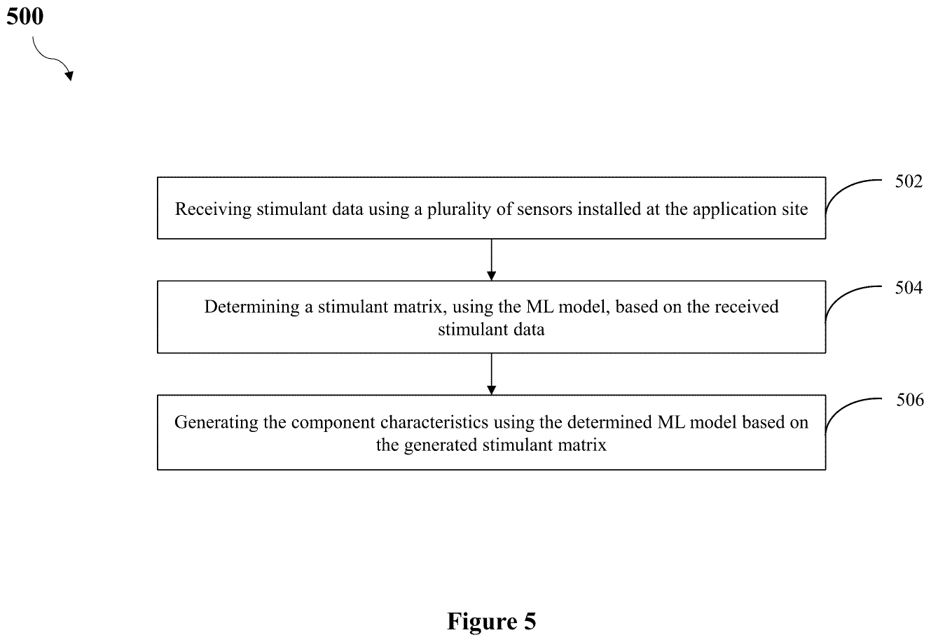

At block 310, the ML module 118 may be configured to generate component characteristics, using the determined ML model. The component characteristics may include physical and chemical features of the one or more components present in the application site. In an embodiment, the component characteristics of the component may correspond to the attribute. The generation of the component characteristics by the ML module 118 is described in greater detail in conjunction with FIG. 5.

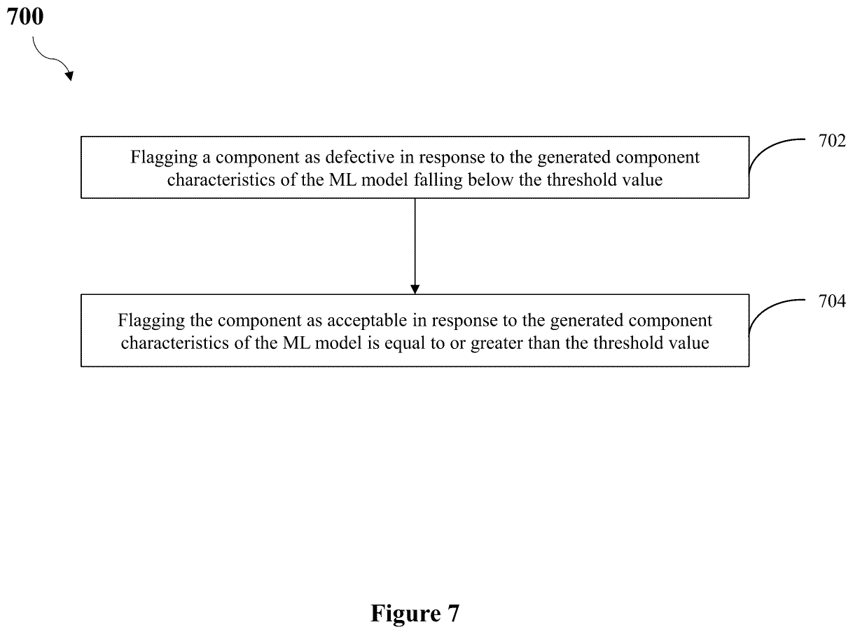

At block 312, the determining module 120 may be configured to determine the one or more defects 132 in the one or more components based on correlating the component characteristics with the threshold value. The one or more defects 132 may indicate deviation or anomaly in the attribute of each of the one or more components. The deviation in the attribute of the component from the threshold value may be flagged as defective or acceptable. In an embodiment, the attribute may include stimuli characteristics for each of the one or more components present in the application site. The one or more defects 132 in the one or more components determined by the determining module 120 is described in greater detail in conjunction with FIG. 7.

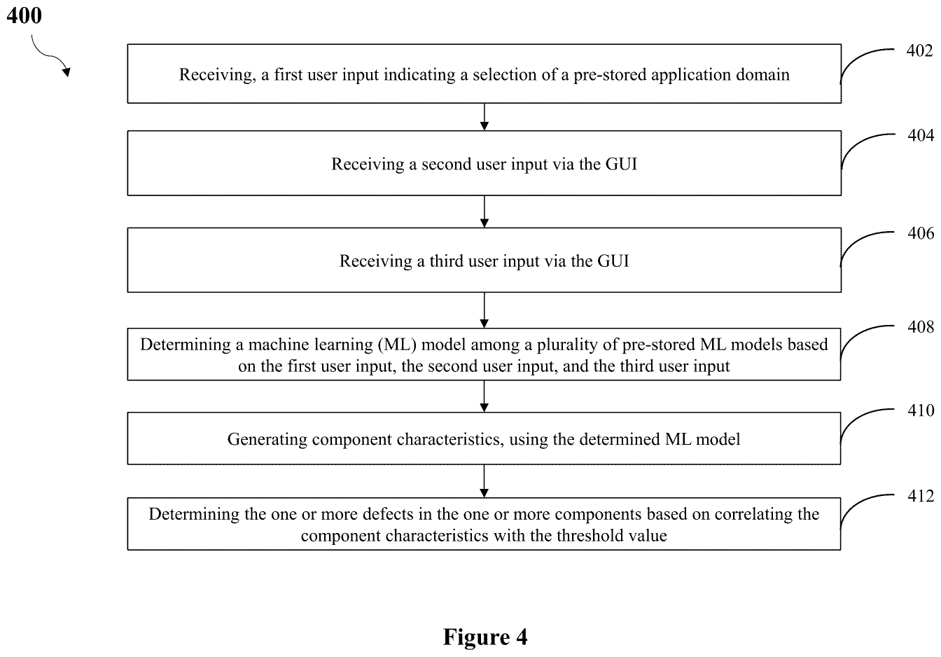

Now, the present disclosure is explained in detail in reference to a method 400 disclosed in FIG. 4. More specifically, referring to FIGS. 1-4 in combination, the various steps of the method 400 as described hereinafter may be executed in the system 104, or specifically in the processor 106 of the system 104, for determining the one or more defects 132 in the one or more components.

FIG. 4 illustrates a flowchart depicting the exemplary method 400 for determining the one or more defects 132 in the one or more components, in accordance with an embodiment of the present disclosure. In an embodiment, the method 400 is a computer-implemented method 400 that is explained in detail in the below paragraphs.

At operation 402, the method 400 may include receiving, the first user input indicating the selection of the pre-stored application domain. The first user input is received via the GUI, as described above.

At operation 404, the method 400 may include receiving the second user input via the GUI. The second user input includes the threshold value associated with the attribute for each of the one or more components.

At operation 406, the method 400 may include receiving the third user input via the GUI. The third user may include the one or more external variables associated with the environment at the application site, as described earlier.

At operation 408, the method 400 may include determining the ML model among the plurality of pre-stored ML models based on the first user input, the second user input, and the third user input.

In an embodiment, the ML model may be determined from the plurality of pre-stored ML models based on an expected performance corresponding to the threshold value. The threshold value may also be referred to as a client threshold or a boundary threshold. For instance, based on the first input A, the second input B, and the third input C, there are three models, Model 1, Model 2, Model 3. For Model 1, the threshold value is 5 for all the A, B, and C: A<10, [0,2,4,6,8]; B<10, [0,2,4,6,8]; and C<10, [0,2,4,6,8]. Similarly, for Model 2, the client threshold or client requirement may be to determine n where the output is 10\hat{ }ⁿ, and the Model 2 may be trained on A [0, 10, 100, 1000]; B [0, 10, 100, 1000]; and C [0, 10, 100, 1000]. Further, Model 3 may be trained with the A, B, C as, A<100,[0,20,40,60,80]; B<100,[0,20,40,60,80]; and C<100,[0,20,40,60,80], and here the boundary threshold may be 50 for all the inputs, A, B, and C.

At operation 410, the method 400 may include generating component characteristics, using the determined ML model. The component characteristics indicate physical and chemical features of the one or more components present in the application site.

In an embodiment, the component may comprise physical and chemical features or elements. For instance, the component is a mixed solution or stimulant of various strengths, for example, 20% water, 20% vodka, and 60% orange juice. Further, the mixed solution is measured through various channels, each having predefined threshold values, for example, channel 1, with predefined threshold value of 100, channel 2 with predefined threshold value of 29, channel 3 with predefined threshold value of 80, channel 4 with predefined threshold value of 90. Further, the stimulant is fed to the ML model, and the ML model predicts strength of each stimulant based on measurement through the various channels. In an embodiment, the various channels may be referred to as the plurality of sensors 102 configured to detect the stimulant data 130. Further, the ML model predicts that the stimulant or the mixed solution is 15% water, 15% vodka and 70% orange juice.

At operation 412, the method 400 may include determining the one or more defects 132 in the one or more components based on correlating the component characteristics with the threshold value. The one or more defects 132 may be deviation or anomaly in the attribute of each of the one or more components. In an embodiment, the component may be flagged as acceptable or defective when the generated component characteristics are above or below the threshold value.

Referring to the above example, the threshold value or the client threshold is that the vodka needs to be at least 10%, therefore, the mixed solution or the stimulant may be flagged as acceptable. Further, the client threshold is that the vodka needs to be at least 20%, therefore, the stimulant is flagged as defective.

In an embodiment, the method 400 may include displaying results over the GUI. The results may include graphic visualizations of stimulant intensities in customizable visual formats for industry-specific reporting of radar plots and bar charts. Further, the GUI displays the threshold value associated with the attribute of each of the one or more components in the radar plots and bar charts. Further, the one or more defect 132 in the generated component characteristics of the ML model may also be displayed over the GUI. In an embodiment, the GUI may display historical trends of the stimulant intensities and variations in the stimulant intensities with respect to time.

In an embodiment, the method 400 may include monitoring remotely, detection parameters, over the GUI, associated with the selection of the ML model and setting of the second user input. The detection parameters may be monitored remotely through any human-computer interface (HCI), for example, local, edge, cloud, etc.

In an embodiment, the method 400 may include storing the threshold value indicative of the attributes for each of the one or more components corresponding to the application site, for subsequent stimuli detection operations.

FIG. 5 illustrates a flowchart 500 depicting sub-steps for generating component characteristics using the ML model, in accordance with an embodiment of the present disclosure.

At sub step 502, the processor 106 may be configured to receive the stimulant data 130 using the plurality of sensor 102 installed at the application site. In an embodiment, the plurality of sensors 102 may be installed at various locations of the application site, for example, in a petroleum refinery, different sensors, such as pressure sensors, motion sensors, flowmeter, gas sensors, flame sensors, etc., may be installed and these sensors may be configured to detect data related to the component, for example, gas. Further, the detected data may be collated in a matrix format and may be referred to as the stimulant data 130.

Further, the processor 106 may be configured to perform a sensor health check by transmitting the data detected by the plurality of sensors 102 to a sensor health check model. The sensor health check model may be referred to as a health monitoring module for the plurality of sensors 102. The sensor health check model may be configured to monitor health of the plurality of sensors 102 by receiving data at periodic intervals from the plurality of sensors 102. For instance, at time tₒ, the processor 106 may transmit some data to the sensor health check model to estimate accuracy of the ML model, and the plurality of sensors at the tₒ, may have an accuracy of 95%. The sensor health check model may predict that the ML model may have an accuracy of 90%, based on the data received from the plurality of sensors 102. Therefore, the plurality of sensors 102 may pass the threshold value, for example, 80%.

Further, at time t₁, the sensor health check model may estimate the accuracy of 75%, and the accuracy is below the threshold value of 80%. In this case, the sensor health check model may indicate to stop further detection. At time t₁, the data detected by the plurality of sensors 102 may change from initial data, for example, a light bulb may have a luminance of 10 at time tₒ. After, 2 months, the luminance may drop to 8 and therefore, the accuracy of the plurality of sensors 102 may drop to 80% which is the threshold value, or commonly referred to as a client threshold.

At sub step 504, the processor 106 may be configured to determine a stimulant matrix, using the ML model, based on the received the stimulant data 130. The stimulant matrix comprises a chemical composition value, a concentration range, and characteristics of compounds. In one example, the stimulant matrix may include an odorant matrix. The odorant matrix includes data related to the odor of the component, e.g., gas, detected by the plurality of sensor 102.

In an embodiment, the stimulant matrix may include datasets determined by the ML model from the stimulant data 130. For example, the stimulant matrix for the stimulant or the mixed solution includes 15% water, 15% vodka and 15% orange juice.

In an exemplary embodiment, different application sites have different stimulants and each of the stimulants may be detected at different resolutions. For instance, for an entire set of stimulants, A, B, C, D, E, . . . , Z, industry 1 typically may focus on 0.001A to 0.01A, 0.1B to 0.5B, while industry 2 may focus on 1A to 3A to 0.0001C to 0.0002C. In this case, the selection of the pre-defined application domain may depend on the stimulant type and resolution associated with that industry/domain.

At sub step 506, the processor 106 may be configured to generate the component characteristics using the determined ML model based on the generated stimulant matrix. As described earlier, in operation 410, the component characteristics may be generated for the stimulant or the mixed solution including 15% water, 15% vodka and 15% orange juice.

FIG. 6 illustrates a flowchart 600 depicting sub-steps for generating an alert in response to an estimated accuracy of the ML model, in accordance with an embodiment of the present disclosure;

At sub step 602, the processor 106 may be configured to estimate the accuracy of the ML model based on supplying the stimulant data 130 at predefined intervals to an accuracy check ML model. Referring to sub step 502, the sensor health check model may be configured to estimate the accuracy of the ML model by feeding some data from the plurality of sensors 102 to the sensor health check model, at period intervals.

At sub step 604, the processor 106 may be configured to generate the alert in response to the estimated accuracy of the ML model below a predefined accuracy threshold for each of the plurality of sensors 102. In an embodiment, the alert may include an anomaly in at least one of the plurality of sensors 102.

In an embodiment, the anomaly is also referred to as a deviation or drift in data from at least one of the plurality of sensors 102. For example, at tₒ, data from the sensor health check model indicates that the accuracy of the ML model is 90%, and at t₁, the accuracy of the ML model is 75%. This deviation of data between the two intervals indicates the anomaly in the sensor.

FIG. 7 illustrates a flowchart 700 depicting sub-steps for determining a defect in the generated output of the ML model, in accordance with an embodiment of the present disclosure;

At sub-step 702, the processor 106 may be configured to flag the component as defective in response to the generated component characteristics of the ML model falling below the threshold value, the component indicates a physical or chemical compound present in the application site.

At sub-step 704, the processor 106 may be configured to flag the component as acceptable in response to the generated component characteristics of the ML model is equal to or greater than the threshold value.

As described in the earlier example, the threshold value or the client threshold is that the vodka needs to be at least 10%, therefore, the mixed solution or the stimulant may be flagged as acceptable. Further, the client threshold is that the vodka needs to be at least 20%, therefore, the stimulant is flagged as defective.

The embodiments disclosed herein can be implemented through at least one software program running on at least one hardware device and performing network management functions to control the elements. The elements can be at least one of a hardware device or a combination of hardware devices and software modules.

It is understood that terms including “unit” or “module” at the end may refer to the unit for processing at least one function or operation and may be implemented in hardware, software, or a combination of hardware and software.

While specific language has been used to describe the disclosure, any limitations arising on account of the same are not intended. As would be apparent to a person in the art, various working modifications may be made to the method in order to implement the inventive concept as taught herein.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein.

Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

The foregoing description of the specific embodiments will so fully reveal the general nature of the embodiments herein that others can, by applying current knowledge, readily modify and adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of at least one embodiment, those skilled in the art will recognize that the embodiments herein can be practiced with modification within the spirit and scope of the embodiments as described herein.

We claim:

1. A method for determining one or more defects in one or more components, the method comprising:
receiving a first user input indicating a selection of a pre-stored application domain, wherein the first user input is received via a graphical-user-interface (GUI);
receiving a second user input via the GUI, the second user input indicates a threshold value associated with an attribute for each of the one or more components;
receiving a third user input via the GUI, the third user indicates one or more external variables associated with an environment at an application site;
determining a machine learning (ML) model among a plurality of pre-stored ML models based on the first user input, the second user input, and the third user input;
generating component characteristics, using the determined ML model, wherein the component characteristics indicate physical and chemical features of the one or more components present in the application site; and
determining the one or more defects in the one or more components based on correlating the component characteristics with the threshold value, the one or more defects indicate deviation or anomaly in the attribute of each of the one or more components.

2. The method as claimed in claim 1, wherein generating the component characteristics using the determined ML model, comprises:
receiving stimulant data using a plurality of sensors installed at the application site;
determining a stimulant matrix, using the ML model, based on the received stimulant data, wherein the stimulant matrix comprises a chemical composition value, a concentration range, and characteristics of compounds; and
generating the component characteristics using the determined ML model based on the generated stimulant matrix.

3. The method as claimed in claim 1, wherein the attribute indicates stimuli characteristics for each of the one or more components present in the application site.

4. The method as claimed in claim 2, comprising:
estimating an accuracy of the ML model based on supplying the stimulant data at predefined intervals to an accuracy check ML model; and
generating an alert in response to the estimated accuracy of the ML model is below a predefined accuracy threshold for each of the plurality of sensors, wherein the alert indicates an anomaly in at least one of the plurality of sensors.

5. The method as claimed in claim 1, wherein the determining defect in the generated output of the ML model comprises:
flagging a component as defective in response to the generated component characteristics of the ML model falling below the threshold value, the component indicates a physical or chemical compound present in the application site; or
flagging the component as acceptable in response to the generated component characteristics of the ML model is equal to or greater than the threshold value.

6. The method as claimed in claim 1, comprising:
displaying, by the GUI, graphic visualizations of stimulant intensities in customizable visual formats for industry-specific reporting of radar plots and bar charts;
displaying, by the GUI, the threshold value associated with the attribute of each of the one or more components in the radar plots and bar charts;
displaying, by the GUI, the one or more defect in the generated component characteristics of the ML model; and
displaying, by the GUI, historical trends of the stimulant intensities and variations in the stimulant intensities with respect to time.

7. The method as claimed in claim 1, comprising:
monitoring remotely detection parameters, over the GUI, associated with the selection of the ML model and setting of the second user input.

8. The method as claimed in claim 1, comprising:
storing the threshold value indicative of the attributes for each of the one or more components corresponding to the application site, for subsequent stimuli detection operations.

9. A system for determining one or more defects in one or more components, the system comprising:
a memory;
a processor communicatively coupled to the memory, the processor is configured to:
receive a first user input indicating a selection of a pre-stored application domain, wherein the first user input is received via a graphical-user-interface (GUI);
receive a second user input via the GUI, the second user input indicates a threshold value associated with an attribute for each of the one or more components;
receive a third user input via the GUI, the third user indicates one or more external variables associated with an environment at an application site;
determine a machine learning (ML) model among a plurality of pre-stored ML models based on the first user input, the second user input, and the third user input;
generate component characteristics, using the determined ML model, wherein the component characteristics indicate physical and chemical features of the one or more components present in the application site; and
determine the one or more defects in the one or more components based on correlating the component characteristics with the threshold value, the one or more defects indicate deviation or anomaly in the attribute of each of the one or more components.

10. The system as claimed in claim 9, wherein the processor to generate the component characteristics using the determined ML model, is configured to:
receive stimulant data using a plurality of sensors installed at the application site;
determine a stimulant matrix, using the ML model, based on the received stimulant data, wherein the stimulant matrix comprises a chemical composition value, a concentration range, and characteristics of compounds; and
generate the component characteristics using the determined ML model based on the generated stimulant matrix.

11. The system as claimed in claim 9, wherein the attribute indicates stimuli characteristics for each of the one or more components present in the application site.

12. The system as claimed in claim 10, wherein the processor is configured to:
estimate an accuracy of the ML model based on supplying the stimulant data at predefined intervals to an accuracy check ML model; and
generate an alert in response to the estimated accuracy of the ML model is below a predefined accuracy threshold for each of the plurality of sensors, wherein the alert indicates an anomaly in at least one of the plurality of sensors.

13. The system as claimed in claim 9, wherein the processor to determine defect in the generated output of the ML model, is configured to:
flag a component as defective in response to the generated component characteristics of the ML model falling below the threshold value, the component indicates a physical or chemical compound present in the application site; or
flag the component as acceptable in response to the generated component characteristics of the ML model is equal to or greater than the threshold value.

14. The system as claimed in claim 9, wherein the processor is configured to:
display, by the GUI, graphic visualizations of stimulant intensities in customizable visual formats for industry-specific reporting of radar plots and bar charts;
display, by the GUI, the threshold value associated with the attribute of each of the one or more components in the radar plots and bar charts;
display, by the GUI, the one or more defect in the generated component characteristics of the ML model; and
display, by the GUI, historical trends of the stimulant intensities and variations in the stimulant intensities with respect to time.

15. The system as claimed in claim 9, wherein the processor is configured to:
monitor remotely detection parameters, over the GUI, associated with the selection of the ML model and setting of the second user input.

16. The system as claimed in claim 9, wherein the processor is configured to:
store the threshold value indicative of the attributes for each of the one or more components corresponding to the application site, for subsequent stimuli detection operations.
